(19) United States
(12) Patent Application Publication
(10) Pub. No.: US 2007/0231842 A1
(43) Pub. Date: Oct. 4, 2007

(54) DETECTION OF AUTOANTIBODIES TO CYTOKERATIN 18 PROTEIN IN PATIENTS WITH BRONCHIAL ASTHMA AND CHRONIC RHINITIS

(76) Inventors: Dong-Ho NAHM, Gyeonggi-Do (KR); Sook-Yeong JEON, Gyeonggi-Do (KR)

Correspondence Address: THE RAFFERTY PATENT LAW FIRM, 5641 BURKE CENTRE PKWY, SUITE 100, BURKE, VA, 22015-2259, US

(21) Appl. No.: 11/563,015

(22) Filed: Nov. 23, 2006

Related U.S. Application Data
(63) Continuation-in-part of application No. 10/514,932, filed on Nov. 12, 2004, now abandoned.

(30) Foreign Application Priority Data
May 15, 2002 (KR) 10-2002-0026765

(51) Int. Cl. G01N 33/564

(52) U.S. Cl. 435/7.92; 436/507

(57) ABSTRACT

The present invention is based on the surprising discovery of autoantibodies to cytokeratin 18 protein in the serum samples of patients with bronchial asthma and chronic rhinitis, especially in nonallergic patients. The present invention includes diagnostic methods and a diagnostic kit to detect patients with bronchial asthma and chronic rhinitis associated with autoantibodies to cytokeratin 18. The invention also includes methods and kits to prescribe or monitor treatment for patients with bronchial asthma and chronic rhinitis by detecting autoantibodies to cytokeratin 18. The present invention also includes a pharmaceutical formulation comprising cytokeratin 18 protein to protect patients with bronchial asthma and chronic rhinitis associated with autoantibodies to cytokeratin 18. The present invention also includes methods to treat patients with bronchial asthma and chronic rhinitis associated with autoantibodies to cytokeratin 18 using compounds that inhibit the interaction between such autoantibodies and cytokeratin 18 protein.

### BACKGROUND OF THE INVENTION

1. Field of the Invention

The present invention relates to diagnostic methods and a diagnostic kit to detect patients with bronchial asthma and chronic rhinitis associated with autoantibodies to cytokeratin 18. More particularly, this invention includes a pharmaceutical formulation comprising cytokeratin 18 protein to protect or treat patients with bronchial asthma and chronic rhinitis associated with autoantibodies to cytokeratin 18. This invention also includes methods to protect or treat patients with bronchial asthma and chronic rhinitis associated with autoantibodies to cytokeratin 18 using compounds that inhibit the interaction between such autoantibodies and cytokeratin 18 protein.

2. Description of the Related Art

Definition and Prevalence of Bronchial Asthma and Chronic Rhinitis

Bronchial asthma is defined as a chronic inflammatory disease of the airways characterized by exacerbations of coughing, wheezing, and difficult breathing that are usually reversible but can be severe and sometimes fatal (National Asthma Education and Prevention Program, NIH publication No. 97-4051, 1997). Bronchial asthma is a common disease, affecting about 5% to 10% of the population in developed countries. Additionally, the prevalence of bronchial asthma has increased over recent decades, probably due to environmental factors (Sears, M. R. Lancet 1997; 350(Suppl2): 1-4.).

Chronic rhinitis is defined as an inflammatory disease of the nasal airway characterized by typical chronic symptoms of rhinorrhea, sneezing, and nasal obstruction (Dykewicz, M. S. et al., Ann Allergy Asthma Immunol 1998; 81:463-468.). Chronic rhinitis is also very common and affects about 10% to 20% of the population in developed countries (Sly, R. M., Ann Allergy Asthma Immunol 1999; 82:233-48).

Bronchial asthma and chronic rhinitis are linked by epidemiologic and immunopathologic characteristics and share some common therapeutic approaches. Most patients with bronchial asthma (about 80-99%) also have chronic rhinitis, and about 30-40% of patients with chronic rhinitis have bronchial asthma as well (Vignola, A. M. et al., Clin Exp Immunol 2001; 31:674-677; Simons, F. E., J Allergy Clin Immunol 1999; 104:534-540). The respiratory epithelium lining the upper and lower airways is histologically similar. From a pathological view point, patients with chronic rhinitis have features of inflammation in the upper airway (nasal mucosa tissue) that are comparable to those in the lower airway (bronchial mucosa tissue) of patients with bronchial asthma. Recently the new term “rhinobronchitis” has been suggested to facilitate appropriate recognition and treatment of the common inflammatory process throughout the upper (rhinitis) and lower airways (asthma) (Simon, F. E., J Allergy Clin Immunol 1999; 104:534-540).

‘Aspirin-exacerbated respiratory disease’ is a clinical syndrome characterized by the presence of chronic rhinitis, nasal polyps, asthma, and the precipitation of both asthma and rhinitis attacks after ingestion of aspirin (Berges-Gimene, M. P. et al., Ann Allergy Asthma Immunol 2002; 89:474-478). The existence of the above syndrome provides evidence that a common pathogenetic mechanism works in both bronchial asthma and chronic rhinitis (Picado, C., Curr Allergy Asthma Rep 2002; 2:488-493).

Etiology and Pathogenetic Mechanism of Bronchial Asthma and Chronic Rhinitis

The primary etiology and mechanism causing the development of bronchial asthma and chronic rhinitis is not yet completely understood (National Asthma Education and Prevention Program, NIH publication No. 97-4051, 1997; Dykewicz, M. S. et al., Ann Allergy Asthma Immunol 1998; 81 :463-468). Traditionally, an allergic immune response to common environmental agents (allergens such as house dust mites and pollens) has been regarded as an important mechanism responsible for the development of airway inflammation in patients with bronchial asthma and chronic rhinitis (Lemanske, R. F., Jr et al., JAMA 1997; 278:1855-1873; Dykewicz, M. S. et al., Ann Allergy Asthma Immunol 1998; 81:463-468.). However, allergic response to common environmental agents cannot be detected in a significant proportion (about 40%-50%) of patients with bronchial asthma and chronic rhinitis (Pearce, N. et al., Thorax 1999; 54:268-272; Settipane, R. A. et al., Ann Allergy Asthma Immunol 2001; 86:494-508). These patients have been classified as having nonallergic asthma and rhinitis. Nonallergic asthma and rhinitis often begin at an older age and are clinically more severe than allergic asthma and rhinitis (Virchow, J. C. Jr. et al., J Allergy Clin Immunol 1996; 98:S27-S33; Settipane, R. A. et al., Ann Allergy Asthma Immunol 2001; 86:494-508). However, the mechanism responsible for the development of airway inflammation in patients with nonallergic asthma and rhinitis cannot be explained yet.

Diagnosis, Classification, and Treatment of Bronchial Asthma and Chronic Rhinitis

The diagnosis of bronchial asthma and chronic rhinitis can be achieved by a characteristic history and objective tests (National Asthma Education and Prevention Program, NIH publication No. 97-4051, 1997; Dykewicz, M. S. et al., Ann Allergy Asthma Immunol 1998; 81:463-468).

Bronchial asthma can be diagnosed when a history of typical clinical symptoms such as intermittent cough, dyspnea, or wheezing is present and reversibility of airway obstruction can be documented by pulmonary function measurements before and after inhalation of a bronchodilator. Demonstration of airway hyper-reactivity to nonspecific stimuli (methacholine or histamine, etc.) also can be objective evidence for the diagnosis of bronchial asthma.

Chronic rhinitis is mainly diagnosed by a history of typical chronic symptoms such as rhinorrhea, sneezing, and nasal obstruction rather than objective laboratory tests. Demonstration of eosinophilic leukocytes in the nasal secretion or nasal mucosa tissue by microscopic examination also can sometimes be helpful for the diagnosis of chronic rhinitis.

In patients with bronchial asthma and chronic rhinitis, the presence of an allergic reaction to common environmental inhalant agents (allergens such as house dust mites and pollens) can be examined by allergy skin test or by in-vitro tests for specific IgE antibodies to allergens in serum samples. The examination of allergic reaction to environmental agents is clinically useful for the identification of environmental risk factors, which can precipitate the exacerbation of bronchial asthma and chronic rhinitis, and also useful for classification of allergic patients with bronchial asthma and chronic rhinitis as differentiated from nonallergic patients with the diseases. However, the examination of allergic reaction to environmental allergens cannot be used for diagnosis of bronchial asthma and chronic rhinitis because positive reaction is also present in about 20%-30% of apparently healthy people and more than 50% of patients with other diseases like atopic dermatitis and allergic conjunctivitis (Pearce, N. et al., Thorax 1999; 54:268-272.).

In allergic patients with bronchial asthma and chronic rhinitis, clinical symptoms can be improved by reducing exposure to sensitized allergens or by reducing the patient's sensitivity to allergens through immunotherapy. During immunotherapy, the allergens are regularly administered hypodermically in order to reduce the allergic reaction to those allergens (National Asthma Education and Prevention Program, NIH publication No. 97-4051, 1997; Dykewicz, M. S. et al., Ann Allergy Asthma Immunol 1998; 81:463-468).

As a pharmacological therapy, corticosteroid has been known to be the most effective medication for the treatment of bronchial asthma and chronic rhinitis. Direct administration of corticosteroid to the target tissue by nasal spray or inhalation devices is preferred method over systemic administration to avoid systemic side effects. For further symptomatic control of bronchial asthma, additional treatment with an inhaled bronchodilator can be useful. Oral administration of antihistamine can also be useful for reducing the symptoms of chronic rhinitis (National Asthma Education and Prevention Program, NIH publication No. 97-4051, 1997; Dykewicz, M. S. et al., Ann Allergy Asthma Immunol 1998; 81:463-468).

The effectiveness of treatment for patients with bronchial asthma and chronic rhinitis can be monitored by tracking the history of changes in clinical symptoms. Serial measurements of objective pulmonary function can also be useful for monitoring the effect of treatment in patients with bronchial asthma.

Problems in Current Definition and Classification of Bronchial Asthma and Chronic Rhinitis

Bronchial asthma and chronic rhinitis are not diseases but syndromes including various heterogeneous diseases regarding etiology, pathogenetic mechanism, and natural history (Rackemann, F. M., J Allergy 1940; 11:147-162; Virchow, J. C. Jr. et al., J Allergy Clin Immunol 1996; 98:S27-S33; Dykewicz, M. S. et al., Ann Allergy Asthma Immunol 1998; 81:478-518; Sobol, S. E. et al., Curr Allergy Asthma Rep 2001; 1:193-201). Etiological classification of bronchial asthma and chronic rhinitis is difficult because the primary etiology of bronchial asthma and chronic rhinitis is not completely understood yet (National Asthma Education and Prevention Program, NIH publication No. 97-4051, 1997; Dykewicz, M. S. et al., Ann Allergy Asthma Immunol 1998; 81:478-518). Current criteria for classification of bronchial asthma and chronic rhinitis are mainly dependent on the examination of allergic reaction to common environmental allergens. And there is no currently available test method for the direct detection of patients with nonallergic asthma and rhinitis except demonstrating the absence of allergic reaction to about 10 to 30 specific common environmental allergens.

Problems in Current Method for Diagnosis of Bronchial Asthma and Chronic Rhinitis

Primary-care physicians mainly depend on the clinical history and physical examination for the diagnosis of bronchial asthma and chronic rhinitis. Objective laboratory tests are not widely used due to the following reasons, and this sometimes results in misdiagnosis or delayed diagnosis of such diseases: pulmonary function measurement requires special equipment and a trained operator; allergy skin testing is accompanied by minor physical discomfort of patients due to a needle prick in the skin and requires a skilled person to administer the test; in-vitro testing for specific IgE antibodies to common allergens also needs special laboratory equipment and a skilled person and usually requires testing for multiple allergens; examination of nasal eosinophilic leukocytes is not routinely used due to lack of consensus on the diagnostic value of this test (Dykewicz, M. S. et al., Ann Allergy Asthma Immunol 1998; 81:463-468); there is no available laboratory diagnostic test for chronic rhinitis with consensus on its diagnostic value.

Pathogenesis and Diagnostic Methods of Other Chronic Inflammatory Diseases

In various kinds of chronic inflammatory diseases such as rheumatoid arthritis, systemic lupus erythematosus, chronic atrophic gastritis, and thyroiditis, the autoimmune response against the self-antigen (autoantigen) is known to play an important role in the pathogenesis of disease. In these diseases, tests for detecting autoantibodies to various autoantigens are widely used for diagnosing and monitoring the diseases, prediction of prognosis, and choice of treatment methods (Davidson, A. et al., N Engl J Med 2001; 345:340-350).

Problems in the application of autoantibody tests for diagnosis and classification of bronchial asthma and chronic rhinitis:

Higher incidences of various autoantibodies against antigens in bronchial mucosa, nasal mucosa, paranasal sinus, lung, and endothelial cells have been reported in patients with bronchial asthma and chronic rhinitis compared to healthy controls, especially in nonallergic patients (Girard, J. P. et al., Poumon Coeur 1973; 29:267-270; Wagner, V. et al., Acta Allergol 1965;20:1-9; Yassin, A. et. Al., J Laryngol Otol 1974;88:39-46; Lassalle, P. et al., Eur J Immunol 1993;23:796-803). On the basis of these studies, previous investigators have suggested that an autoimmune mechanism might be involved in the pathogenesis of bronchial asthma and chronic rhinitis. However, previous studies could not establish a causal relationship between autoimmunity and asthma due to the lack of an identified autoantigen or lack of a logical association between these autoantibodies and airway inflammation. Autoantibody testing is not currently used for the diagnosis or classification of bronchial asthma and chronic rhinitis.

Problems in Current Treatment Methods for Bronchial Asthma and Chronic Rhinitis

Because the primary etiology and mechanism causing the development of bronchial asthma and chronic rhinitis is not completely understood yet, a treatment method that can induce complete remission of bronchial asthma and chronic rhinitis has not been developed yet. Current pharmacological therapy can improve the clinical symptoms and physiological functions only during the continuous administration of medication and is not yet proven to modify the long-term natural course of bronchial asthma and chronic rhinitis (National Asthma Education and Prevention Program, NIH publication No. 97-4051, 1997; Dykewicz, M. S. et al., Ann Allergy Asthma Immunol 1998; 81:478-518).

### SUMMARY OF THE INVENTION

The present invention is based on the surprising discovery of autoantibodies to cytokeratin 18 protein in serum samples of patients with bronchial asthma and chronic rhinitis, especially in nonallergic patients. The inventors believe this to be the first reporting of such autoantibodies associated with patients with bronchial asthma and chronic rhinitis.

The inventors went to great effort to demonstrate the presence of autoantibodies to airway epithelial cell proteins in the bodily fluid of patients with bronchial asthma and chronic rhinitis, and to identify the autoantigen reacting with such autoantibodies. After exhaustive experiments to screen and purify the autoantigen, the inventors finally identified the cytokeratin 18 protein as the airway epithelial cell autoantigen associated with bronchial asthma and chronic rhinitis, especially in nonallergic patients. The present invention relates to application of cytokeratin 18 protein for the diagnosis and classification of patients with bronchial asthma and chronic rhinitis. The present invention also includes a pharmaceutical formulation comprising cytokeratin 18 protein and methods to protect or treat patients with bronchial asthma and chronic rhinitis using compounds that inhibit the interaction between autoantibodies and cytokeratin 18.

### BRIEF DESCRIPTION OF THE DRAWINGS

FIG. 1 shows immunoblot analysis of IgG autoantibodies to human airway epithelial cell (BEAS-2B) antigens in serum samples from healthy controls (lane 1-3), patients with allergic asthma and rhinitis (lane 4-7), patients with nonallergic asthma and rhinitis (lane 8-11), patients with systemic lupus erythematosus (lane 12-14), a patient with nonallergic asthma and rhinitis as a positive control (lane 15), and dilution buffer only as a negative control (lane 16). * Arrow indicates the 49-kDa autoantigen.

FIG. 2 shows the protein staining of purified 49-kDa autoantigen separated by SDS-PAGE. Protein staining shows molecular weight standard (lane 1), whole cell extract of airway epithelial cell (BEAS-2B) (lane 2), 49-kDa autoantigen purified by ion-exchange chromatography and reverse-phase HPLC (lane 3), and purified bovine cytokeratin 18 protein (lane 4).

FIG. 3 shows the chromatograph of peptide fragments derived from trypsin-digestion of purified 49-kDa autoantigen separated by reverse-phase HPLC. Two fractions of peptide fragments (peaks A and B) were sequenced.

FIG. 4 shows immunoblot analysis of IgG autoantibodies in serum samples from two patients with nonallergic asthma and rhinitis and a monoclonal antibody to cytokeratin 18. Whole cell extract of airway epithelial cells (BEAS-2B) (lanes 1, 4, and 7), purified 49-kDa autoantigen (lanes 2, 5, and 8), and purified bovine cytokeratin 18 (lanes 3, 6, and 9) were subjected to immunoblot analysis. Autoantibodies in serum samples from two patients with nonallergic asthma and rhinitis (lanes 1-3, lane 7-9) and monoclonal antibody to cytokeratin 18 (lanes 4-6) recognized the purified 49-kDa autoantigen and purified bovine cytokeratin 18.

FIG. 5 shows immunoblot analysis of IgG autoantibodies to human cytokeratin 18 protein in serum samples from healthy controls (lane 1, 2), a patient with allergic asthma and rhinitis (lane 3), and patients with nonallergic asthma and rhinitis (lane 4-6). A monoclonal antibody to cytokeratin 18 was used as a positive control (lane 7) and dilution buffer only was used as a negative control (lane 8). *Arrow indicates the cytokeratin 18 protein.

FIG. 6 shows detection of IgG autoantibodies to purified human cytokeratin 18 protein in serum samples from 2 patients with nonallergic asthma and rhinitis and a pooled serum of 10 healthy controls by enzyme-linked immunosorbent assay (ELISA).

FIG. 7 shows immunoblot analysis of IgA autoantibodies to cytokeratin 18 protein in serum samples from a healthy control (lane 1), patients with allergic asthma and rhinitis (lane 2-4), and patients with nonallergic asthma and rhinitis (lane 5-10). A monoclonal antibody to cytokeratin 18 was used as a positive control (lane 11). *Arrow indicates the cytokeratin 18 protein.

FIG. 8 shows complement-mediated cytotoxicity to airway epithelial cells (BEAS-2B) in serum samples from healthy controls (group 1), patients with allergic asthma and rhinitis who have no detectable IgG autoantibodies to cytokeratin 18 (group 2), patients with nonallergic asthma and rhinitis who have no detectable IgG autoantibodies to cytokeratin 18 (group 3), and patients with nonallergic asthma and rhinitis who have IgG autoantibodies to cytokeratin 18 (group 4).

FIG. 9 shows complement-mediated cytotoxicity to airway epithelial cells (BEAS-2B) in the pooled serum sample of patients who have IgG autoantibodies to cytokeratin 18 (Serum only) and inhibition of the cytotoxicity by addition of purified human cytokeratin 18 protein (Serum+CK18) or human serum albumin (Serum+HSA). The data were obtained from 8 individual experiments and expressed as mean value and standard deviation.

FIG. 10 shows immunoblot analysis of IgG autoantibodies to purified human cytokeratin 18 protein in serum samples of allergic asthma patients without clinical evidence of chronic rhinitis (lane 1-3) and nonallergic asthma patients without clinical evidence of chronic rhinitis (lane 4-7). A monoclonal antibody to cytokeratin 18 was used as a positive control (lane 8). *Arrow indicates the cytokeratin 18 protein.

FIG. 11 shows immunoblot analysis of IgG autoantibodies to cytokeratin 18 protein in serum samples of allergic rhinitis patients without bronchial asthma (lane 1-3), nonallergic rhinitis patients without bronchial asthma (lane 4-7), and healthy controls (lane 9, 10). A monoclonal antibody to cytokeratin 18 was used as a positive control (lane 8). *Arrow indicates the cytokeratin 18 protein.

### DETAILED DESCRIPTION OF THIS INVENTION

The inventors believe that the autoimmune response against the airway epithelial cell protein induces the development of airway inflammation in a certain proportion of patients with bronchial asthma and chronic rhinitis, especially in nonallergic patients, on the basis of the following reasons.

(1) Airway epithelium has been suggested as a target for the inflammatory response in bronchial asthma and chronic rhinitis on the basis of pathological studies (Montefort, S. et al., Clin Exp Allergy 1992; 22:511-520; Vignola, A. M. et al., Clin Exp Immunol 2001; 31:674-677; Wladislavosky-Waserman, P. et al., Clin Allergy 1984; 14:241-247).

(2) Presence of autoantibodies to bronchial mucosa tissue has been reported in patients with bronchial asthma (Girard, J. P. et al., Poumon Coeur 1973; 29:267-270.; Wagner, V. et al., Acta Allergol 1965; 20:1-9) and autoantibodies to nasal mucosa tissue have been reported in patients with chronic rhinitis (Yassin, A. et al., J Laryngol Otol 1974; 88:39-46.). And both bronchial and nasal mucosa tissues are lined by the same type of respiratory epithelial cells.

(3) Analysis of bronchial tissue samples from patients with adult-onset asthma and from patients who had died in severe asthmatic attack (status asthmaticus) demonstrated depositions of IgG antibodies and complement in the bronchial epithelium and in the cytoplasm of the bronchial epithelial cells (Molina, C. et al., Clin Allergy 1977; 7: 137-45; Callerame, M. L. et al., N Eng J Med 1971; 284: 459-64).

(4) Autoantibodies to cytokeratin 18 from the bodily fluid of patients with bronchial asthma and chronic rhinitis can damage airway epithelial cells through autoantibody-dependent complement-mediated cytotoxicity as disclosed herein.

(5) Removal of plasma containing autoantibodies from a patient with severe asthma induced clinical improvement (Lassalle, P. et al., Clin Exp Allergy 1990; 20:707-712.). Intravenous administration of immunoglobulin from healthy donors decreased the requirement of systemic corticosteroid treatment in patients with severe asthma (Salmun, L. M. et al., J Allergy Clin Immunol 1999; 103:810-815.).

The inventors believe that autoantibodies in the bodily fluid of patients with bronchial asthma and chronic rhinitis can react with airway epithelial cell protein in the upper and lower airway (nasal and bronchial mucosa). Such autoantibody-autoantigen immune complexes can induce chronic inflammation of the upper and lower airway by the complement-mediated cytotoxicity and activation of inflammatory cells. Then, chronic inflammation of the airway can induce the clinical features of bronchial asthma and chronic rhinitis.

The present invention relates to a method for the diagnosis of bronchial asthma and chronic rhinitis, including the steps of (a) obtaining a bodily fluid from a subject suspected of having bronchial asthma and chronic rhinitis, (b) contacting the bodily fluid with cytokeratin 18 protein under conditions suitable for the formation of an immune complex between cytokeratin 18 protein and autoantibodies to cytokeratin 18, and (c) determining the presence of autoantibodies to cytokeratin 18 by detecting said immune complex, wherein the presence of said immune complex indicates that the subject has bronchial asthma and chronic rhinitis. Techniques to determine the presence of such an immune complex between autoantigen and autoantibodies are known to those skilled in the art, examples of which are disclosed herein. Disclosure of such techniques can be found, for example, in Rose et al., Manual of Clinical Laboratory Immunology, American Society for Microbiology Press, 1997. A bodily fluid can include any fluid collectible from a human subject such as, but not limited to, blood, serum, plasma, urine, tears, saliva, nasal secretion, bronchial secretion, lung secretion, and any other secretions.

As used herein, a cytokeratin 18 protein refers to any mammalian cytokeratin 18 or a fragment thereof, such that the fragment retains the ability to bind to the autoantibodies to cytokeratin 18 in bodily fluid from patients with bronchial asthma and chronic rhinitis. A cytokeratin 18 protein can either be isolated or expressed from cells, tissues or microorganisms and can be produced using standard methods in the art, including but not limited to recombinant DNA technology. Human cytokeratin 18 protein consists of 430 amino acids, and the sequence has been reported [reference: Oshima, R. G., Millan, J. L., Cecena, G. Comparison of mouse and human keratin 18: a component of intermediate filaments expressed prior to implantation. Differentiation 1986; 33: 61-68]. The sequence number 1 (SEQ ID NO: 1) is the amino acid sequence of human cytokeratin 18 protein. Mouse cytokeratin 18 protein consists of 423 amino acids, and the sequence has been reported [Ichinose, Y., Morita, T., Zhang, F. Y., Srimahasongcram, S., Tondella, M. L., Matsumoto, M., Nozaki, M., Matsushiro, A. Nucleotide sequence and structure of the mouse cytokeratin endoB gene. Gene 1988; 70:85-95]. The sequence number 2 (SEQ ID NO: 2) is the amino acid sequence of mouse cytokeratin 18 protein. Cytokeratin 18 is a cytoskeletal protein found primarily in epithelial cells lining the respiratory and gastrointestinal tracts, including bronchial epithelial cells and lung (alveolar) epithelial cells (Moll, R. et al., Cell 1982; 31:11-24). Although cytokeratin 18 is a predominantly intracellular protein, its strong expression on the cell surface was also observed in epithelial cells (Moll, R. et al., Cell 1982; 31:11-24; Saarloos, M. N. et al., Curr Eye Res 1999; 19:439-449).

The present invention includes a method to detect nonallergic patients with bronchial asthma and chronic rhinitis, including the steps of (a) obtaining a bodily fluid from a subject suspected of having bronchial asthma and chronic rhinitis, (b) contacting the bodily fluid with cytokeratin 18 protein under conditions suitable for the formation of an immune complex between cytokeratin 18 protein and autoantibodies to cytokeratin 18, and (c) determining the presence of autoantibodies to cytokeratin 18 by detecting said immune complex, wherein the presence of said immune complex indicates that the subject has nonallergic asthma and rhinitis.

The present invention provides a method to aid in the detection of nonallergic asthma and rhinitis in a human subject, comprising the steps of: (a) obtaining a bodily fluid sample from the human subject; (b) contacting the bodily fluid sample with the human cytokeratin 18 protein to form an immune complex between the human cytokeratin 18 protein and the autoantibodies in the bodily fluid sample; and (c) determining the presence of the autoantibodies against the human cytokeratin 18 protein in the bodily fluid sample by detecting the immune complex, wherein the presence of the autoantibodies bears a positive correlation with the existence of nonallergic asthma and rhinitis in the human subject.

The present invention also includes a method to detect or classify patients with bronchial asthma and chronic rhinitis associated with autoantibodies to cytokeratin 18, including the steps of (a) obtaining a bodily fluid from a subject suspected of having bronchial asthma and chronic rhinitis or a patient with bronchial asthma and chronic rhinitis, (b) contacting the bodily fluid with cytokeratin 18 protein under conditions suitable for the formation of an immune complex between cytokeratin 18 protein and autoantibodies to cytokeratin 18, and (c) determining the presence of autoantibodies to cytokeratin 18 by detecting said immune complex, wherein the presence of said immune complex indicates that the subject or patient has bronchial asthma and chronic rhinitis associated with autoantibodies to cytokeratin 18.

The present invention provides a method for detecting bronchial asthma and chronic rhinitis associated with autoantibodies against the human cytokeratin 18 protein in a human subject diagnosed as having bronchial asthma and chronic rhinitis, comprising the steps of: (a) obtaining a bodily fluid sample from the human subject diagnosed as having bronchial asthma and chronic rhinitis; (b) contacting the bodily fluid sample with the human cytokeratin 18 protein to form an immune complex between the human cytokeratin 18 protein and the autoantibodies in the bodily fluid sample; and (c) determining the presence of the autoantibodies against the human cytokeratin 18 protein in the bodily fluid sample by detecting the immune complex, wherein the presence of the autoantibodies indicates the existence of bronchial asthma and chronic rhinitis associated with the autoantibodies against the human cytokeratin 18 protein in the human subject and the presence of the autoantibodies bears a positive correlation with the existence of nonallergic asthma and rhinitis associated with the autoantibodies against the human cytokeratin 18 protein in the human subject.

The present invention also includes a diagnostic kit comprising mammalian cytokeratin 18 protein for detection and classification of patients with bronchial asthma and chronic rhinitis. Said kit includes mammalian cytokeratin 18 protein or fragments thereof and a means to detect autoantibodies to cytokeratin 18 in bodily fluid from human subjects. The assay methods applied to said kit are known to those skilled in the art, examples of which are disclosed herein. In said kit, the assay method includes any techniques that can detect an antigen-antibody reaction such as agglutination immunoassays, light-scattering immunoassays, enzyme-linked immunoassays, radioimmunoassays, fluorescence immunoassays, chemiluminescence immunoassays, immunofixation, and immunoblotting but is not limited to these.

The present invention also includes a method to prescribe the treatment for bronchial asthma and chronic rhinitis, in that the present invention teaches methods to identify bronchial asthma and chronic rhinitis associated with autoantibodies to cytokeratin 18. More specifically, the present invention provides a method for prescribing a treatment for nonallergic asthma and chronic rhinitis associated with the autoantibodies against the human cytokeratin 18 protein to a human subject diagnosed as having bronchial asthma and chronic rhinitis, comprising the steps of: (a) obtaining a bodily fluid sample from the human subject diagnosed as having bronchial asthma and chronic rhinitis; (b) contacting the bodily fluid sample with the human cytokeratin 18 protein to form an immune complex between the human cytokeratin 18 protein and the autoantibodies in the bodily fluid sample; (c) determining the presence of the autoantibodies against the human cytokeratin 18 protein in the bodily fluid sample by detecting the immune complex, wherein the presence of the autoantibodies bears a positive correlation with the existence of nonallergic asthma and rhinitis associated with the autoantibodies against the human cytokeratin 18 protein in the human subject; and (d) prescribing the treatment for nonallergic asthma and chronic rhinitis associated with the autoantibodies against the human cytokeratin 18 protein to the human subject based on the correlation identified in the step (c). (e) The presence of the autoantibodies to the human cytokeratin 18 protein bears a positive correlation with clinical improvement of bronchial asthma and chronic rhinitis in the human subject by treatments with human immunoglobulin or the human cytokeratin 18 protein. Therefore, the step (e) is also described as predicting a clinical response (improvement) to treatments with human immunoglobulin or the human cytokeratin 18 protein in the human subject.

Furthermore, the present invention includes a method to monitor the efficacy of a treatment for patients with bronchial asthma and chronic rhinitis, by periodically detecting autoantibodies to cytokeratin 18 in the bodily fluid of such patients. The present invention also includes kits comprising mammalian cytokeratin 18 for detection of autoantibodies against cytokeratin 18 to prescribe treatments and monitor the efficacy of treatment in patients with bronchial asthma and chronic rhinitis.

The present invention includes a pharmaceutical formulation comprising mammalian cytokeratin 18 or fragments thereof to protect or treat patients with bronchial asthma and chronic rhinitis who have autoantibodies to cytokeratin 18 in their bodily fluids. Such a formulation is designed for the protection of cytokeratin 18-expressing cells from the cytotoxic effect by autoantibodies in the bodily fluid of patients with bronchial asthma and chronic rhinitis. The present invention also includes a pharmaceutical formulation comprising a compound that inhibits the binding between autoantibodies to cytokeratin 18 and cytokeratin 18 protein. Such an inhibitory compound includes whole mammalian cytokeratin 18 protein or fragments thereof which retain an ability to bind to autoantibodies against cytokeratin 18.

The present invention includes a method to protect or treat patients with bronchial asthma and chronic rhinitis who have autoantibodies to cytokeratin 18 protein in their bodily fluids. Such a method includes the step of inhibiting the binding between autoantibodies to cytokeratin 18 and cytokeratin 18-expressing cells by administering an inhibitory compound comprising mammalian cytokeratin 18. Such an inhibitory compound includes whole mammalian cytokeratin 18 protein or fragments thereof retaining an ability to bind to autoantibodies against cytokeratin 18.

The present invention also includes a method to identify a pharmaceutical compound capable of inhibiting the binding of autoantibodies from patients with bronchial asthma and chronic rhinitis to cytokeratin 18 protein or cytokeratin 18-expressing cells. Such an inhibitory compound can be identified by the following steps: (a) contacting the autoantibodies isolated from bodily fluid of patients with bronchial asthma and chronic rhinitis with a putative inhibitory compound; and (b) determining whether the compound can inhibit the binding of such autoantibodies to cytokeratin 18 protein or inhibit the cytotoxic effect of autoantibodies to cytokeratin 18-expressing cells.

The following specific examples are intended to be illustrative of the invention and should not be construed as limiting the scope of the invention as defined by appended claims.

### EXAMPLES

Materials and Methods

Subjects

The inventors examined serum samples from 27 patients with allergic asthma and rhinitis, 23 patients with nonallergic asthma and rhinitis, 34 age-matched healthy controls, and 20 patients with systemic lupus erythematosus. All patients with bronchial asthma and chronic rhinitis had typical clinical histories compatible with bronchial asthma and chronic rhinitis and documented reversibility of forced expiratory volume in one second (FEVI) greater than 15% after inhalation of a bronchodilator or a 20% decrease in FEV₁ following the inhalation of less than 8 mg/ml of methacholine. All patients with bronchial asthma and chronic rhinitis underwent skin-prick tests with 50 common aeroallergens (Bencard Co., Brentford, UK). Patients were classified as having allergic asthma and rhinitis when the wheal diameter of any one allergen was greater than 3 mm over the negative control (normal saline) and there was a definite history or objective evidence of clinical aggravation induced by allergen exposure. Patients with nonallergic asthma and rhinitis showed no positive skin reaction to any of the 50 common aeroallergens, and serum total IgE concentrations were within the normal range (less than 180 IU/ml). Twenty patients with systemic lupus erythematosus classified according to the 1982 revised criteria of the American Rheumatic Association were included as control subjects with disease. All serum samples from subjects were stored at −20° C.

Culture of Airway Epithelial Cells

Human airway epithelial cell lines including BEAS-2B (ATCC CRL-9609; Ke, Y. et al., Differentiation 1988; 38:60-6) and A549 (ATCC CCL-185; Giard, D. J. et al., J Natl Cancer Inst 1973; 51:1417-23) cells were obtained from American Type Culture Collection (ATCC; Manassas, Va.).

Cell Lysis and Protein Extraction

Cultured cells were lysed by the addition of a lysis buffer containing 10 mM Tris/HCl, pH 7.2, 1% Triton X-100, 1% sodium deoxycholate, 0.1% sodium dodecyl sulphate, 158 mM NaCl, 1 mM EGTA, 1 mM Na₃VO₄, 250 μg/ml leupeptin and ImM phenylmethylsulfonyl fluoride.

Immunoblot Analysis

Proteins in cell lysates were separated by discontinuous sodium dodecyl sulphate/polyacrylamide gel electrophoresis (SDS-PAGE) using an 8% resolving gel (pH 8.8) and a 4% stacking gel (pH 6.8). Following electrophoresis, proteins were transferred onto a polyvinylidine difluoride membrane (PVDF; Bio-Rad Laboratories, Hercules, Calif.). After the transfer, the PVDF membrane was blocked with Tris-buffered saline (TBS) containing 10% bovine serum and 0.1% TWEEN-20™ polyethylene glycol sorbitan monolaurate). The PVDF membrane strips were then probed with 1 ml serum samples at 1:100 dilution for 2 hours at room temperature. After washing, the membrane was incubated with alkaline phosphatase-conjugated goat anti-human IgG (Sigma Chemical Co., St. Louis, Mo.) for 2 hours at room temperature. After a final wash, the membrane was stained with a BCIP/NBT solution (5-bromo-4-chloro-3-indoyl phosphate/nitro blue tetrazolium; Sigma). To confirm the identification of 49-kDa autoantigen after the amino acid sequence analysis, a mouse monoclonal antibody to human cytokeratin 18 (clone no. CY-90, Sigma) and a negative control mouse monoclonal antibody with the same IgGI isotype (Sigma Chemical Co., St. Louis, Mo.) were used for immunoblot analysis. Alkaline phosphatase-conjugated goat anti-mouse IgG (Sigma Chemical Co., St. Louis, Mo.) was used as a secondary conjugate and the results were developed as above. Commercially available purified bovine cytokeratin 18 protein (Research Diagnostics INC., Pleasant Hill Road Flanders, N.J.) was used as a positive control antigen in the experiment using mouse monoclonal antibody to human cytokeratin 18.

Purification and Identification of Autoantigen

For purification of the autoantigen, airway epithelial cell (BEAS-2B) lysates were fractionated by ion-exchange chromatography with diethylaminoethyl (DEAE) Sepharose bead (Sigma Chemical Co., St. Louis, Mo.). Fractions of interest were analyzed by SDS-PAGE and immunoblot analysis, further concentrated with CENTRIPREP-50™ (Amicon, Witten, Germany) and subjected to reverse-phase high-performance liquid chromatography (HPLC) using VYDAC™ C18 column (The Separation Group, Inc., Hesperia, Calif.). Fractions were collected and lyophilized. They were examined by SDS-PAGE and immunoblot analysis. Because analysis of the purified protein on PVDF revealed that the N-terminal amino acid sequence was blocked, the protein was subjected to enzymatic in-gel digestion by trypsin. Trypsin-digested peptide fragments were separated by a micro-HPLC system using SEPHASIL™ C18 reverse-phase column (Amersham Pharmacia Biotech, Uppsala, Sweden). Two fractions of peptide fragments were subjected to amino acid sequencing using Procise cLC 492 Protein sequencing system (Applied Biosystems, Foster, Calif.). To compare the amino acid sequences of peptide fragments with known protein sequences, the SWISS-PROT database (Swiss Institute of Bioinformatics, Geneva, Switzerland; The European Bioinformatics Institute, Cambridge, U.K.) was used.

Detection of Other Autoantibodies

All of the serum samples were also tested for IgG antinuclear antibodies (ANA), and IgG autoantibodies to thyroglobulin and thyroid peroxidase. IgG antinuclear antibodies were assessed by an indirect immunofluorescence staining of HEp2 cells (Hemagen Diagnostics Inc., Maryland, USA), and IgG autoantibodies to thyroglobulin and thyroid peroxidase were measured by radioimmunoassay (BRAHMS DIAGNOSTICA GMBH, Berlin, Germany).

Complement-Mediated Cytotoxicity to Airway Epithelial Cells by Autoantibodies

The complement-mediated cytotoxicity to airway epithelial cells by autoantibodies was measured using 3-(4,5-dimethylthiazol-2-yl)-2,5-diphenyltetrazolium bromide (MTT). The experiment was conducted using serum samples from 8 patients with nonallergic asthma and rhinitis who have IgG autoantibodies to cytokeratin 18, 6 patients with nonallergic asthma and rhinitis who have no detectable IgG autoantibodies to cytokeratin 18, 8 patients with allergic asthma and rhinitis who have no detectable IgG autoantibodies to cytokeratin 18, and 8 healthy controls. The airway epithelial cell (BEAS-2B) was cultured on the 96 well culture plate. When the cells covered 50% of the surface of each well, the cytotoxicity was measured. Before the measurement of cytotoxicity, the serum samples were treated at 56° C. for 30 minutes to inactivate the complement. After the culture medium was removed from each well, 100 μl of triplicated serum samples diluted 1:20 in DMEM/F12 medium were added to each well or DMEM/F12 medium only was added to each well as a control. The plate was incubated for 90 minutes at 37° C. in the CO₂ incubator. Then, the 10 μl of rabbit complement was added to each well and the mixture was incubated for 90 minutes at 37° C. in the CO₂ incubator. After culture medium including serum sample and complement was removed from each well, 200 μl of fresh DMEM/F12 medium including 10% fetal bovine serum and 50 μl of the MTT solution was added to each well and the plate was incubated for 3 hours at 37° C. in the CO₂ incubator. Then the supernatant was removed from each well and 200 μl of dimethyl sulfoxide was added to each well. Finally, 25 μl of 0.1 M glycine, 0.1 M NaCl buffer (pH 10.5) was added to each well. Absorbance at 570 nm was measured by microplate reader. Complement-mediated cytotoxicity was expressed as % cell lysis using the following formula with absorbance values of test wells including serum samples and control wells including the culture medium only. Cytotoxicity (% cell lysis)=[(absorbance of control wells−absorbance of test wells)/absorbance of control wells]×100.

Inhibition of Complement-Mediated Cytotoxicity to Airway Epithelial Cells

The same volumes of serum samples from 8 patients with nonallergic asthma and rhinitis who have IgG autoantibodies to cytokeratin 18 were mixed and made into a pooled serum sample. This pooled serum was treated for 30 minutes at 56° C. and diluted 1:20 in DMEM/F12 medium and the complement-mediated airway epithelial cytotoxicity was measured. To inhibit a complement-mediated cytotoxicity to airway epithelial cells in the pooled serum, purified human cytokeratin 18 protein or human serum albumin was added to the diluted pooled serum at the 100 μg/ml final concentration of inhibitors. The mixture was incubated for 2 hours at 37° C. and then complement-mediated cytotoxicity was measured. The results were obtained from 8 individual experiments and expressed as a mean value and standard deviation.

Results

Detection of IgG Autoantibodies to Bronchial Epithelial Cell Antigen

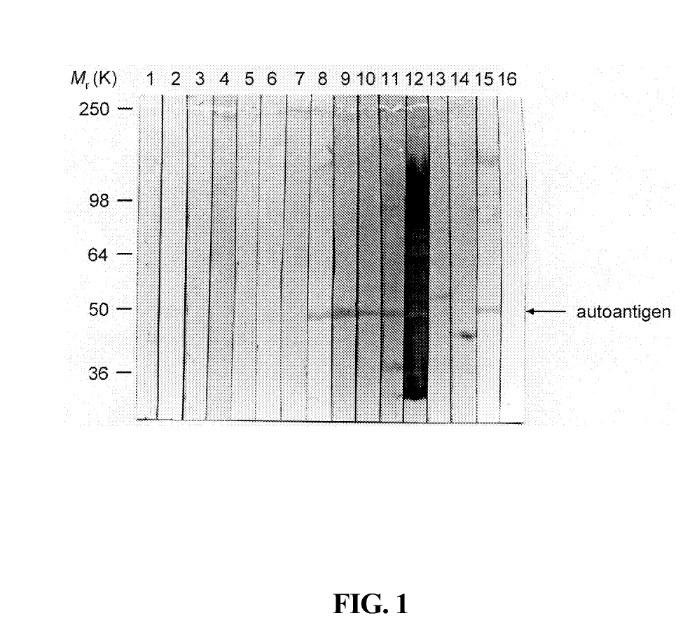

IgG autoantibodies to 49-kDa bronchial epithelial cell antigen were detected in serum samples from 10 of 23 patients with nonallergic asthma and rhinitis (43%), 3 of 27 patients with allergic asthma and rhinitis (11%), 2 of 20 patients with systemic lupus erythematosus (10%), and 3 of 34 age-matched healthy controls (9%) (FIG. 1, Table 1; chi-square test, p<0.005). The positive rate of IgG autoantibodies to 49-kDa bronchial epithelial cell antigen was significantly higher in patients with nonallergic asthma and rhinitis compared to patients with allergic asthma and rhinitis, patients with systemic lupus erythematosus, and healthy controls (Table 1; Fisher's exact test, p<0.05). Therefore, it could be recognized that the sensitivity and specificity of IgG autoantibodies to the cytokeratin 18 protein for the discrimination of nonallergic asthma and rhinitis from other diseases (allergic asthma and rhinitis, and systemic lupus erythematosus) and normal control were about 43% and 90%, respectively. This specificity value (about 90%) indicates that the autoantibody assay of the present invention has a significant diagnostic value, considering that this specificity value of the present invention is much higher than that (68.3%) of the antinuclear antibody test, the most valuable blood test for the diagnosis of systemic lupus erythematosus (Tan E M, et al., Arthritis Rheum. 1997; 40:1601-11). In addition, other autoantibody assays patented as diagnostic methods for asthma in the U.S.A. exhibit the sensitivity and/or specificity lower than or similar to the present invention using autoantibodies to the cytokeratin 18 protein. For example, U.S. Pat. No. 6,165,799 titled as “Detection of Anti-Fc_{ε}RI Autoantibodies in Asthmatics” shows 40% sensitivity to non-atopic intrinsic asthma (see Example 1) even without any verification about the specificity of the autoantibody assay for excluding other diseases. U.S. Pat. No. 5,861,264 discloses anti-tryptase detection as a diagnostic method for inflammatory diseases shows about just 78% specificity.

Given the autoantibody assays described above (U.S. Pat. No. 6,165,799 and U.S. Pat. No. 5,861,264) that are considered meaningful and useful diagnostic methods for bronchial asthma, it is evident that the sensitivity and specificity values evaluated in this Example render the autoantibody assay of the present invention to be significantly useful in the detection of nonallergic asthma and rhinitis.

Table 1 shows the detection rate of IgG autoantibodies to the 49-kDa airway epithelial cell antigen in patients with allergic asthma and rhinitis, patients with nonallergic asthma and rhinitis, patients with systemic lupus erythematosus, and healthy controls.

TABLE 1

| | | Autoantibodies | |
|---|---|---|---|
| Groups | Number | positive (%) | p* |
| Healthy controls | 34 | 3 (9%) | 0.002 |
| Allergic asthma and rhinitis | 27 | 3 (11%) | 0.009 |
| Systemic lupus erythematosus | 20 | 2 (10%) | 0.015 |
| Nonallergic asthma and rhinitis | 23 | 10 (43%) | |

*A statistical significance of the difference between two groups (nonallergic asthma and rhinitis versus other group) was calculated using Fisher's exact test.

Detection of Other IgG Autoantibodies

The positive rates of IgG antinuclear antibodies and IgG autoantibodies to thyroid autoantigens were not significantly different among patients with nonallergic asthma and rhinitis, patients with allergic asthma and rhinitis, and healthy controls (Table 2, p>0.05).

Table 2 shows the detection rate of IgG antinuclear antibodies, IgG autoantibodies to thyroglobulin, and IgG autoantibodies to thyroid peroxidase in patients with allergic asthma and rhinitis, patients with nonallergic asthma and rhinitis, patients with systemic lupus erythematosus, and healthy controls.

TABLE 2

Autoantibodies positive (%)

| Groups | Number | ANA | Anti-TG | Anti-TPO |
|---|---|---|---|---|
| Healthy controls | 34 | 1 (3%) | 1 (3%) | 2 (6%) |
| Allergic asthma and | 27 | 0 (0%) | 1 (4%) | 2 (7%) |
| rhinitis | | | | |
| Nonallergic asthma and | 23 | 3 (13%) | 2 (9%) | 0 (0%) |
| rhinitis | | | | |
| Systemic lupus | 20 | 19 (95%)* | 3 (15%) | 3 (15%) |
| erythematosus | | | | |

ANA: antinuclear antibodies,
Anti-TG: anti-thyroglobulin antibodies,
Anti-TPO: anti-thyroid peroxidase antibodies.
*A significant statistical difference compared with 3 other groups (p < 0.05).

Purification and Identification of 49-kDa Airway Epithelial Cell Autoantigen

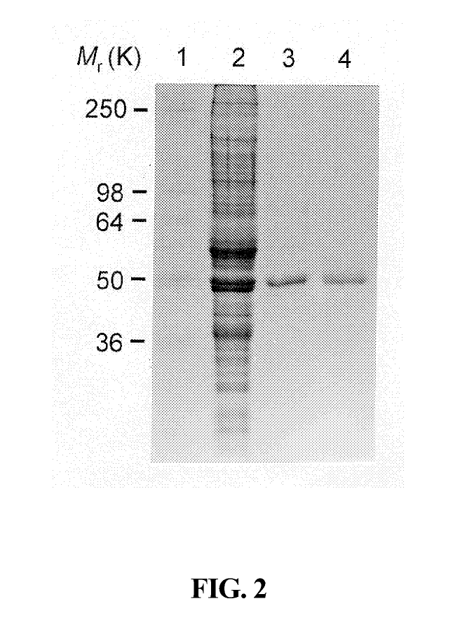

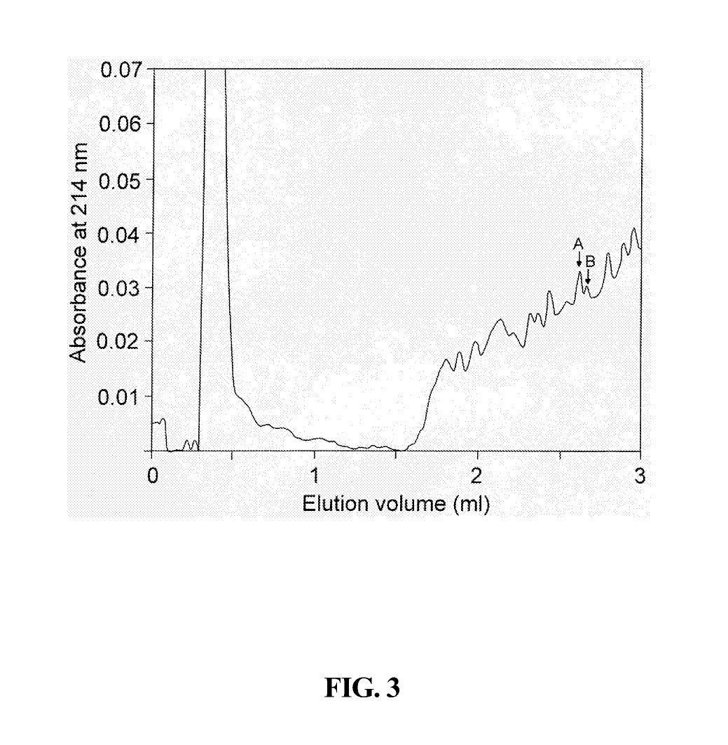

To characterize the 49-kDa airway epithelial cell autoantigen, this protein was purified by ion-exchange chromatography and reverse-phase HPLC. The purified protein was separated in an 8% tris-glycine gel (FIG. 2). The purified protein was then subjected to enzymatic in-gel digestion by trypsin, and the peptide fragments were separated by reverse-phase HPLC (FIG. 3). The two fractions (peak A and peak B) of peptide fragments were subjected to amino acid sequencing. It was found that the amino acid sequences of two peptide fragments (SEQ ID NO:3 and SEQ ID NO:4) matched accurately with those of the human cytokeratin 18 protein (SEQ ID NO:1) upon database analysis as shown in Table 3.

Table 3 shows the amino acid sequences of two peptide fragments of the purified 49-kDa autoantigen and the matched amino acid sequences of the human cytokeratin 18 protein (SEQ ID NO:1) and mouse cytokeratin 18 protein (SEQ ID NO:2) in the database.

TABLE 3

| Peptide fragment 1 | Trp Ser His Tyr Phe Lys |
|---|---|
| (SEQ ID NO:3) | 1 5 |
| Human cytokeratin 18 protein | Trp Ser His Tyr Phe Lys |
| (residues 126 to 131 of SEQ ID:1) | 126 130 |
| Peptide fragment 2 | Leu Glu Ala Glu Ile Ala Thr Tyr Arg |
| (SEQ ID NO:4) | 1 5 |
| Human cytokeratin 18 protein | Leu Glu Ala Glu Ile Ala Thr Tyr Arg |
| (residues 373 to 381 of SEQ ID:1) | 373 375 380 |
| Mouse cytokeratin 18 protein | Leu Glu Ala Glu Ile Ala Thr Tyr Arg |
| (residues 366 to 374 of SEQ ID:2) | 366 370 |

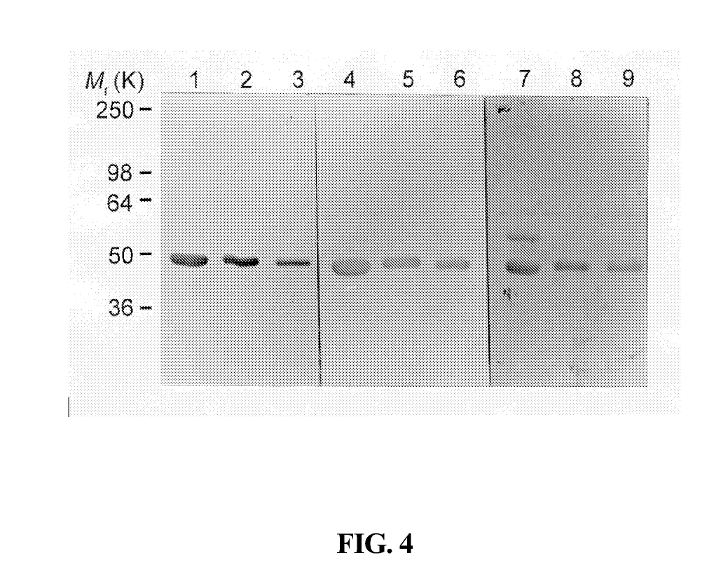

The identification of the 49-kDa airway epithelial cell autoantigen as human cytokeratin 18 was further confirmed by immunoblot analysis using a monoclonal antibody against human cytokeratin 18 and comparing with purified bovine cytokeratin 18 protein (FIG. 4).

Complement-Mediated Cytotoxicity to Airway Epithelial Cells by Autoantibodies

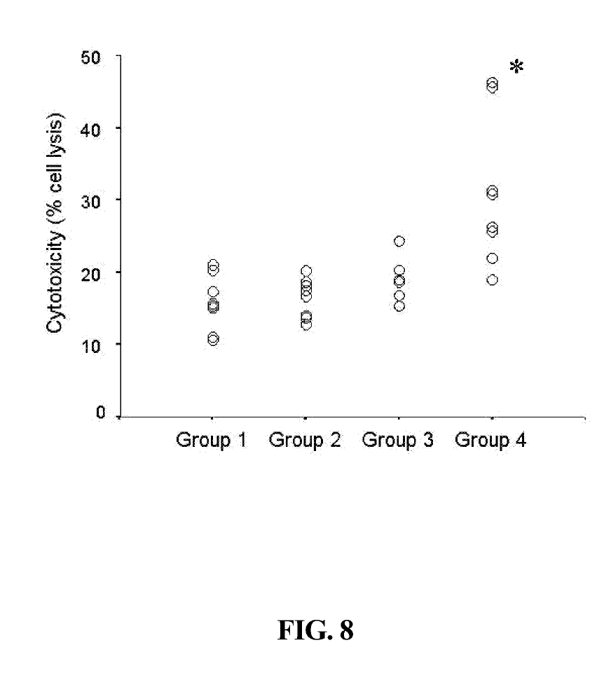

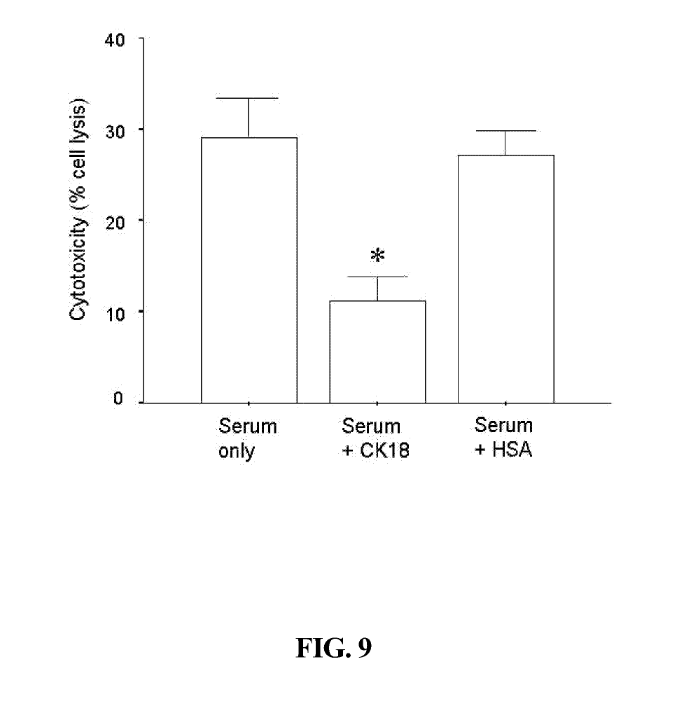

Complement-mediated cytotoxicity to airway epithelial cells was significantly higher in the serum samples of patients with nonallergic asthma and rhinitis who have IgG autoantibodies to cytokeratin 18 (mean±standard deviation; 30.9±10.2%) than patients with nonallergic asthma and rhinitis who have no detectable IgG autoantibodies to cytokeratin 18 (19.1±3.1%), patients with allergic asthma and rhinitis who have no detectable IgG autoantibodies to cytokeratin 18 (16.5±2.7%), and healthy controls (15.8±3.8%) (FIG. 8, p<0.005). Complement-mediated cytotoxicity to airway epithelial cells was not detectable when only the heat-inactivated serum samples were added without the complement. Moreover, complement-mediated cytotoxicity to airway epithelial cells in the pooled serum sample (29.1±4.3%) was significantly inhibited by addition of the purified human cytokeratin 18 protein (11.3±2.6%) but not by addition of human serum albumin (27.3±2.7%) (FIG. 9, p<0.005). These results demonstrate that airway epithelial cells can be damaged by autoantibodies to cytokeratin 18 present in the serum samples of patients with nonallergic asthma and rhinitis through complement-mediated cytotoxicity. Furthermore, a significant inhibition of complement-mediated cytotoxicity to airway epithelial cells by purified human cytokeratin 18 protein clearly demonstrates that human cytokeratin 18 protein can protect the airway epithelial cells from damage by autoantibodies in bodily fluid from patients with bronchial asthma and chronic rhinitis who have autoantibodies to cytokeratin 18 protein. And this result also indicates that administration of human cytokeratin 18 protein can protect those patients with bronchial asthma and chronic rhinitis who have autoantibodies to cytokeratin 18 from the airway epithelial cell damage caused by circulating autoantibodies.

### Example 1

### Detection of IgG and IgA Autoantibodies to Cytokeratin 18 in Serum Samples from Patients with Bronchial Asthma and Chronic Rhinitis by Immunoblot Analysis

Whole cell extract from human airway epithelial cells (A549 cell) or purified human cytokeratin 18 protein was separated by SDS-PAGE (4% stacking gel and 8% running gel), and the protein was transferred onto the PVDF membrane. The PVDF membrane was incubated with TBS containing 5% nonfat dried milk and 0.05% TWEEN-20™ (blocking buffer) for 1 hour to prevent nonspecific protein bindings to the PVDF, then the membrane was made to 4 mm-wide strips. The PVDF strips were incubated with serum samples diluted 1:100 in blocking buffer for 2 hours at room temperature. After washing, the PVDF strips were incubated with alkaline phosphatase-conjugated goat anti-human IgG or anti-human IgA antibodies for 2 hours. After washing, the PVDF strips were stained with BCIP/NBT substrate solution for 5 minutes. As a positive control, one PVDF strip was incubated with mouse monoclonal antibody to cytokeratin 18 instead of human serum sample and alkaline phosphatase-conjugated goat anti-mouse IgG antibodies were used as a secondary conjugate and stained with BCIP/NBT. As a negative control, one PVDF strip was incubated with blocking buffer only instead of serum samples.

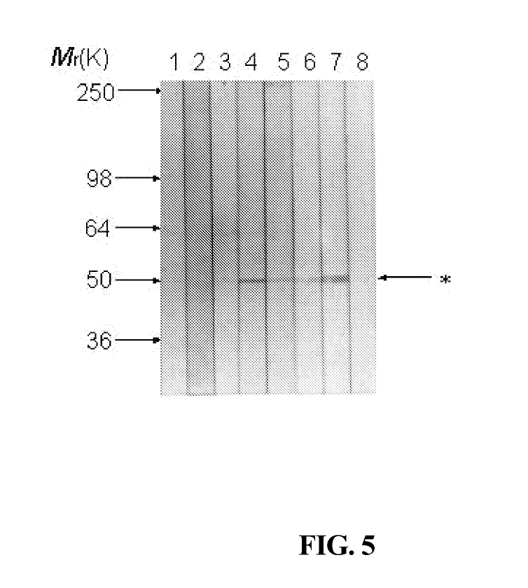

Upon the immunoblot analysis of IgG autoantibodies to cytokeratin 18 in serum samples using whole cell extract of human airway epithelial cells (A549), IgG autoantibody to cytokeratin 18 was negative in 2 healthy controls and a patient with allergic asthma and rhinitis and positive in 3 patients with nonallergic asthma and rhinitis (FIG. 5).

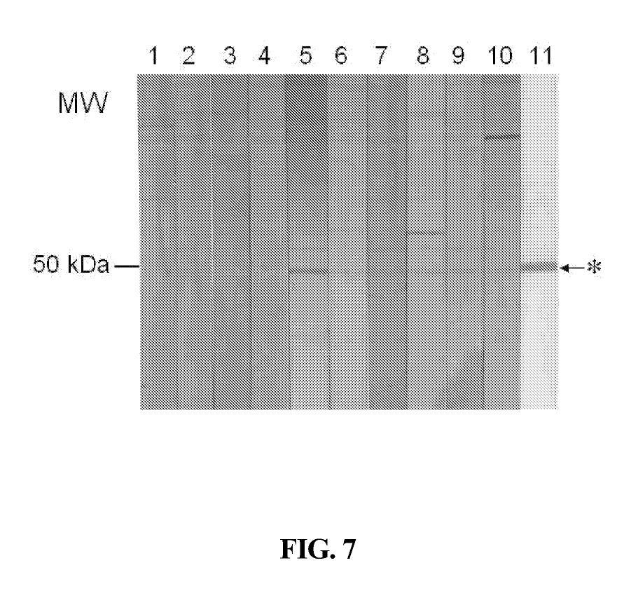

Upon the immunoblot analysis of IgA autoantibodies to cytokeratin 18 in serum samples using whole cell extract of human airway epithelial cells (A549), IgA autoantibody to cytokeratin 18 was negative in a healthy control and 3 patients with allergic asthma and rhinitis and positive in 6 patients with nonallergic asthma and rhinitis (FIG. 7).

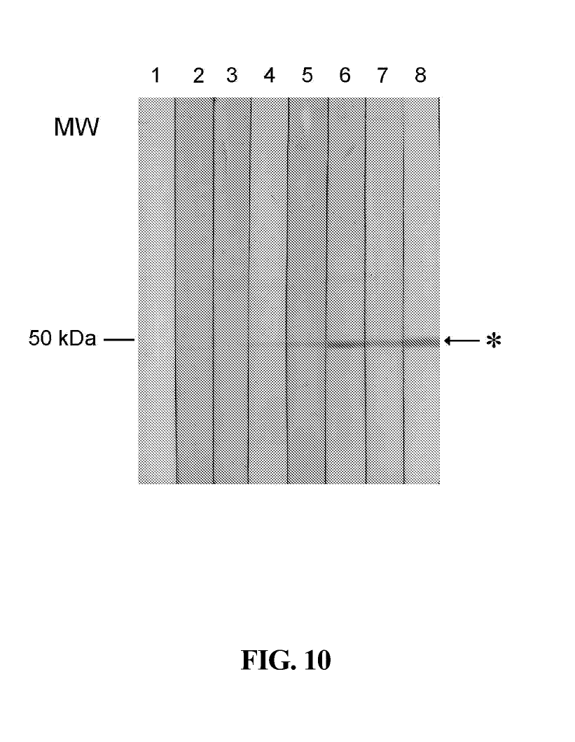

Upon the immunoblot analysis of IgG autoantibodies to cytokeratin 18 in serum samples using purified human cytokeratin 18 protein, IgG autoantibody to cytokeratin 18 was negative in 3 allergic asthma patients without clinical evidence of chronic rhinitis and positive in 4 nonallergic asthma patients without clinical evidence of chronic rhinitis (FIG. 10).

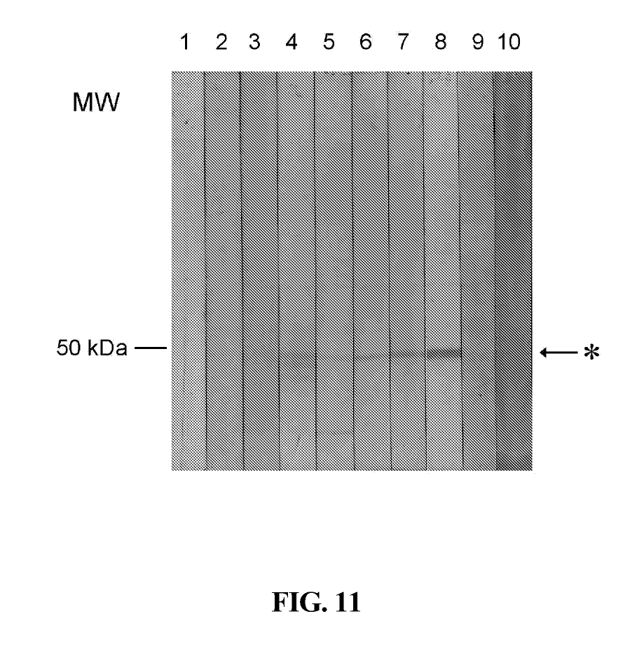

Upon the immunoblot analysis of IgG autoantibodies to cytokeratin 18 in serum samples using whole cell extract of human airway epithelial cells (A549), IgG autoantibody to cytokeratin 18 was negative in 2 healthy controls and 3 allergic rhinitis patients without bronchial asthma and positive in 4 nonallergic rhinitis patients without bronchial asthma (FIG. 11).

### Example 2

### Detection of IgG Autoantibodies to Cytokeratin 18 in Serum Samples from Patients with Bronchial Asthma and Chronic Rhinitis by Enzyme-Linked Immunosorbent Assay (ELISA)

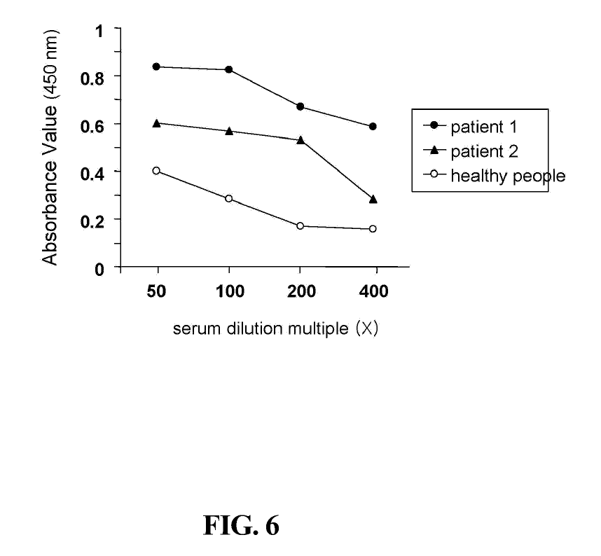

Microtiter plates were coated with purified human cytokeratin 18 protein at a concentration of 0.5 μg per well in 0.1M carbonate buffer (pH 9.6) for 16 hours at 4° C. After washing 3 times with phosphate buffered saline containing 0.05% TWEEN-20™ (PBST), wells were incubated with 350 μl of PBST containing 3% fetal bovine serum for 1 hour at room temperature. After washing 3 times with PBST, wells were incubated with 100 μl of quadruplicated serum samples diluted in PBST containing 3% fetal bovine serum for 2 hours. After washing 3 times, wells were incubated with peroxidase-conjugated goat anti-human IgG antibodies (Sigma) for 2 hours. After washing 3 times, 100 μl of the TMB substrate solution (Sigma) was added to each well. After 10 minutes, the reaction was stopped by adding 100 μl of 2.5 N H₂SO₄ to each well. The absorbance was measured at 450 nm using an ELISA reader. Absorbance values from serum samples of 2 patients with nonallergic asthma and rhinitis were significantly higher than the absorbance values from a pooled serum sample of 10 healthy controls (FIG. 6).

### Example 3

### Method to Prescribe Treatment for Nonallergic Asthma and Rhinitis by Detection of IgG Autoantibodies to Cytokeratin 18 in Serum Samples

Although several non-steroidal immunomodulatory drugs such as intravenous immunoglobulin (Salmun L M, et al. J Allergy Clin Immunol 1999; 103:810-5), cyclosporine, gold, methotrexate, and hydroxychloroquine have been reported to be beneficial to severe asthmatic patients, their use in asthma remains complicated because of highly variable effects in individual patients and the absence of a marker predicting responsiveness to such treatments (Frew A J, et al. J Allergy Clin Immunol 2001; 108:3-10).

Here, the present invention shows a method to prescribe intravenous immunoglobulin for patients with severe asthma on the basis of detection of IgG autoantibodies to cytokeratin 18 in the serum samples.

Two adult patients with nonallergic asthma and rhinitis were admitted to the hospital due to severe aggravation of their asthmatic symptoms. The two patients received standard therapy for exacerbation of asthma including high dose intravenous corticosteroid therapy (62.5 mg of methyl prednisolone per 6 hours) and maximal doses of nebulized bronchodilator (salbutamol) with nasal oxygen supply for 5 days. However, their asthmatic symptoms and pulmonary functions did not improve. After informed consent, a high dose of intravenous immunoglobulin (0.4 g/kg/day) was administered to the two patients for 2 days (admission day 6, 7) along with continuation of standard therapy. After intravenous immunoglobulin therapy, patient 1 showed dramatic clinical improvement of asthmatic symptoms and objective pulmonary function parameters but patient 2 did not show significant improvement (Table 4).

Table 4 shows changes of asthma severity in two patients with nonallergic asthma and rhinitis who were admitted to the hospital because of asthma exacerbation. Asthma severity was expressed as peak expiratory flow rate (PEFR) that was the mean value of 3 measurements at 7:00 am before the use of inhaled bronchodilator.

TABLE 4

| Admission day | | | | | | | | | |
|---|---|---|---|---|---|---|---|---|---|
| 1 | 2 | 3 | 4 | 5 | 6* | 7* | 8 | 9 | 10 |

Patient 1

| | | | | | | | | | | |
|---|---|---|---|---|---|---|---|---|---|---|
| PEFR (L/min) | 155 | 143 | 162 | 144 | 155 | 135 | 352 | 384 | 411 | 405 |

Patient 2

| | | | | | | | | | | |
|---|---|---|---|---|---|---|---|---|---|---|
| PEFR (L/min) | 191 | 213 | 181 | 193 | 187 | 205 | 220 | 183 | 208 | 228 |

*Intravenous immunoglobulin (0.4 g/kg/day) was administered to patients on admission day 6 and 7.

Immunoblot detection of IgG autoantibodies to cytokeratin 18 in serum samples taken on day 1 showed a positive result in patient 1 (FIG. 4, lanes 1-3) and a negative result in patient 2. These results indicate that detection of IgG autoantibodies to cytokeratin 18 in serum samples from patients with bronchial asthma and chronic rhinitis can be used as a marker predicting responsiveness to immunomodulatory treatment including intravenous immunoglobulin therapy.

The present invention can be used for screening of patients with bronchial asthma and chronic rhinitis by a simple blood test detecting autoantibodies to cytokeratin 18 instead of the complex steps of clinical evaluation and laboratory tests. The present invention also can be used for the detection of nonallergic patients with bronchial asthma and chronic rhinitis showing an autoimmune phenomenon by detecting autoantibodies to cytokeratin 18. The present invention can also be used for the classification of patients with bronchial asthma and chronic rhinitis showing an autoimmune phenomenon by detecting autoantibodies to cytokeratin 18. The present invention can be used to prescribe a specific treatment for patients with bronchial asthma and chronic rhinitis by detecting autoantibodies to cytokeratin 18.

The present invention can be used for a pharmaceutical formulation comprising cytokeratin 18 protein or fragments thereof to protect patients with bronchial asthma and chronic rhinitis associated with autoantibodies to cytokeratin 18. The present invention also can be used to identify a pharmaceutical compound capable of inhibiting the binding ability of autoantibodies to cytokeratin 18 from patients with bronchial asthma and chronic rhinitis to cytokeratin 18 protein or cytokeratin 18-expressing cells.

What is claimed is:

1. A method to aid in the detection of nonallergic asthma and rhinitis in a human subject, comprising the steps of:
(a) obtaining a bodily fluid sample from the human subject;
(b) contacting the bodily fluid sample with the human cytokeratin 18 protein to form an immune complex between the human cytokeratin 18 protein and the autoantibodies in the bodily fluid sample; and
(c) determining the presence of the autoantibodies against the human cytokeratin 18 protein in the bodily fluid sample by detecting the immune complex, wherein the presence of the autoantibodies bears a positive correlation with the existence of nonallergic asthma and rhinitis in the human subject.

2. The method according to claim 1, wherein the human cytokeratin 18 protein has the amino acid sequence consisting of SEQ ID NO:1.

3. The method according to claim 1, wherein the bodily fluid sample is blood, serum or plasma.

4. The method according to claim 3, wherein the bodily fluid sample is serum.

5. The method according to claim 1, wherein the method is carried out in accordance with an immunoblot assay or ELISA (enzyme-linked immunosorbent assay).

6. The method according to claim 1, wherein the autoantibodies are IgG autoantibodies to the human cytokeratin 18 protein.

7. A method for detecting bronchial asthma and chronic rhinitis associated with autoantibodies against the human cytokeratin 18 protein in a human subject diagnosed as having bronchial asthma and chronic rhinitis, comprising the steps of:
(a) obtaining a bodily fluid sample from the human subject diagnosed as having bronchial asthma and chronic rhinitis;
(b) contacting the bodily fluid sample with the human cytokeratin 18 protein to form an immune complex between the human cytokeratin 18 protein and the autoantibodies in the bodily fluid sample; and
(c) determining the presence of the autoantibodies against the human cytokeratin 18 protein in the bodily fluid sample by detecting the immune complex, wherein the presence of the autoantibodies indicates the existence of bronchial asthma and chronic rhinitis associated with the autoantibodies against the human cytokeratin 18 protein in the human subject and the presence of the autoantibodies bears a positive correlation with the existence of nonallergic asthma and rhinitis associated with the autoantibodies against the human cytokeratin 18 protein in the human subject.

8. The method according to claim 7, wherein the human cytokeratin 18 protein has the amino acid sequence consisting of SEQ ID NO:1.

9. The method according to claim 7, wherein the bodily fluid sample is blood, serum or plasma.

10. The method according to claim 9, wherein the bodily fluid sample is serum.

11. The method according to claim 7, wherein the method is carried out in accordance with an immunoblot assay or ELISA (enzyme-linked immunosorbent assay).

12. The method according to claim 7, wherein the autoantibodies are IgG autoantibodies to the human cytokeratin 18 protein.

13. A method for prescribing a treatment for nonallergic asthma and rhinitis associated with autoantibodies against the human cytokeratin 18 protein to a human subject diagnosed as having bronchial asthma and chronic rhinitis, comprising the steps of:
(a) obtaining a bodily fluid sample from the human subject diagnosed as having bronchial asthma and chronic rhinitis;
(b) contacting the bodily fluid sample with the human cytokeratin 18 protein to form an immune complex between the human cytokeratin 18 protein and the autoantibodies in the bodily fluid sample;
(c) determining the presence of the autoantibodies against the human cytokeratin 18 protein in the bodily fluid sample by detecting the immune complex, wherein the presence of the autoantibodies bears a positive correlation with the existence of nonallergic asthma and rhinitis associated with the autoantibodies against the human cytokeratin 18 protein in the human subject; and
(d) prescribing the treatment for nonallergic asthma and rhinitis associated with the autoantibodies against the human cytokeratin 18 protein to the human subject based on the correlation identified in the step (c).

14. The method according to claim 13, wherein the human cytokeratin 18 protein has the amino acid sequence consisting of SEQ ID NO:1.

15. The method according to claim 13, wherein the bodily fluid sample is blood, serum or plasma.

16. The method according to claim 15, wherein the bodily fluid sample is serum.

17. The method according to claim 13, wherein the treatment is an administration of human immunoglobulin.

18. The method according to claim 13, wherein the treatment is an administration of the human cytokeratin 18 protein.

19. The method according to claim 13, wherein the steps (b) and (c) are carried out in accordance with an immunoblot assay or ELISA (enzyme-linked immunosorbent assay).

20. The method according to claim 13, wherein the autoantibodies are IgG autoantibodies to the human cytokeratin 18 protein.


SEQUENCE LISTING (4 sequences)

SEQ ID NO: 1 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 2 - protein, 423 aa, Mus musculus
MSFTTRSTTFSTNYRSLGSVRTPSQRVRPASSAASVYAGAGGSGSRISVSRSVWGGSVGSAGLAGMGGIQTEKETMQDLNDRLASYLDKVKSLETENRRLESKIREHLEKKGPQGVRDWGHYFKIIEDLRAQILANSVDNARIVLQIDNARLAADDFRVKYETELAMRQSVESDIHGLRKVVDDTNITRLQLETEIEALKEELLFMKKNHEEEVQGLEAQIASSGLTVEVDAPKSQDLSKIMADIRAQYEALAQKNREELDKYWSQQIEESTTVVTTKSAEIRDAETTLTELRRTLQTLEIDLDSMKNQNINLENSLGDVEARYKAQMEQLNGVLLHLESELAQTRAEGQRQAQEYEALLNIKVKLEAEIATYRRLLEDGEDFSLNDALDSSNSMQTVQKTTTRKIVDGRVVSETNDTRVLRH

SEQ ID NO: 3 - protein, 6 aa, Homo sapiens
WSHYFK

SEQ ID NO: 4 - protein, 9 aa, Homo sapiens
LEAEIATYR